(19) United States
(12) Patent Application Publication
(10) Pub. No.: US 2015/0157370 A1
(43) Pub. Date: Jun. 11, 2015

(54) SEGMENTAL RECONSTRUCTIVE INTRAMEDULLARY NAIL AND DELIVERY SYSTEM

(71) Applicant: IMPETUS INNOVATIONS, INC., Halifax (CA)

(72) Inventor: Michael Gross, Halifax (CA)

(73) Assignee: IMPETUS INNOVATIONS, INC., Halifax, NS (CA)

(21) Appl. No.: 14/413,489

(22) PCT Filed: Sep. 23, 2013
(86) PCT No.: PCT/CA2013/000800

§ 371 Date: Jan. 8, 2015

Related U.S. Application Data
(60) Provisional application No. 61/704,546, filed on Sep. 23, 2012.

(51) Int. Cl. A61B 17/72; A61B 17/88; A61M 31/00

(52) U.S. Cl. CPC A61B 17/7283; A61M 31/002; A61B 17/8811; A61B 2017/561

(57) ABSTRACT

An intramedullary device, delivery system, surgical method for implanting the device, and a method of assembling the device are disclosed. The intramedullary device includes a nail with a first segment proximate a distal end, a second segment proximate a proximal end, and a delivery segment connecting the first and second segments. The delivery system including a nail and a dispersion device. The nail including at least one first segment, at least one second segment, and a delivery system between the at least one first and second segments. The dispersion device configured to slidingly engage the nail. The surgical method may include inserting an intramedullary device into a canal within a bone and dispensing a biomedical material to the bone. The method of assembling an intramedullary device may include selecting a first segment, delivery segment, and second segment and securing the delivery segment between the first segment and second segment.

### CROSS-REFERENCE TO RELATED APPLICATION

This application claims priority benefit under 35 U.S.C. §119(e) to U.S. provisional application No. 61/704,546 filed Sep. 23, 2012, which is incorporated herein by reference in its entirety.

### FIELD OF THE INVENTION

The present invention relates generally to an intramedullary device and, in particular, to an intramedullary nail for delivering materials to the site of a fracture.

### BACKGROUND

Intramedullary (“IM”) nails are currently used in orthopaedics to reconstruct bones without major defects. Most of the IM nail implants used are static in their function and provide mechanical stability to bone that then heals around or under the implant. Generally IM nails do not do well in the presence of significant bone defects, where there is no mechanical continuity and bone grafting is necessary. Soft tissue injuries are now routinely treated with free flaps by plastic surgeons, however bone grafting is limited to what can be taken from the iliac wings and there are usually inadequate amounts available to fill large defects. Allograft bones are not usually used in potentially infected wounds, and cortical allografts take a long time to be incorporated and become capable of physiologic load bearing activity. In trauma situations with large bone defects, particularly in the tibia, surgeons are conditioned to amputation if there are also associated major soft tissue defects. Amputation is currently used on limbs with devastating soft tissue injuries and segmental bone loss even if there is an intact distal innervation, neurovascular bundle, or nerve in the foot, allowing for a sensate foot. There is a need for an IM nail for use in bones with major defects whether or not there are major soft tissue defects. The major impediment to reconstructing missing bone has been stabilization of the injured limb, soft tissue reconstruction and the methods to deliver and grow new bone while maintaining mechanical stability of the injured limb.

### SUMMARY

The present disclosure relates generally to an intramedullary device with a delivery system for delivering materials to the site of a bone deficiency, due to cancer, significant trauma, bone loss or weakness due to various different clinical conditions, to stimulate bone formation and provide a scaffold for bone formation.

In one aspect, provided herein is an intramedullary device including a nail with a proximal end and a distal end. The nail has a first segment proximate the distal end, a second segment proximate the proximal end, and a delivery segment connecting the first segment and the second segment.

In another aspect, provided herein is a delivery system including a nail and a dispersion device. The nail has a proximal end and a distal end and includes at least one first segment that is proximate to the distal end, at least one second segment that is proximate to the proximal end, and a delivery segment connecting the at least one first segment and the at least one second segment. The dispersion device includes a proximal end and a distal end and is configured to slidingly engage the nail.

In yet another aspect, provided herein is an intramedullary device system that has a nail and a dispersion device. The nail with a proximal end and a distal end includes a first segment at the distal end, a second segment at the proximal end, and a delivery segment positioned medial to the first segment and the second segment. The nail also has a first plurality of extension segments connecting the first segment and the delivery segment and a second plurality of extension segments connecting the delivery segment and the second segment. The dispersion device is configured to transport biomedical material to be dispersed into a bone through an interior channel in the nail.

In another aspect, provided herein is a surgical method for implanting an intramedullary device. The surgical method includes obtaining an intramedullary device. The intramedullary device includes a nail with a proximal end and a distal end and a dispersion device with a proximal end and a distal end. The nail has a first segment at the distal end, a second segment at the proximal end, a delivery segment connecting the first and second segments, and an interior channel extending through the first segment, delivery segment, and second segment. The dispersion device is configured to engage the delivery segment. The nail of the intramedullary device is then inserted into a canal created within a bone. The delivery segment is aligned with a damaged portion of the bone. The dispersion device is then inserted into the interior channel of the nail until the distal end of the dispersion device is aligned with the distal end of the delivery segment. Then a biomedical material is dispensed through the dispersion device and delivery segment to the damaged portion of the bone.

In a further aspect of the present invention, a method of assembling the intramedullary device is disclosed. The method of assembling the intramedullary device includes selecting a first segment. Next a delivery segment is selected and secured to the proximal end of the first segment. A second segment then selected and the second segment is secured on a proximal end of the delivery segment opposite the first segment.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention.

FIG. 1 is an isometric view of an intramedullary device from a distal end, in accordance with an aspect of the present invention;

FIG. 2 is an isometric view of the intramedullary device of FIG. 1 from a proximal end, in accordance with an aspect of the present invention;

FIG. 3 is a side view of the intramedullary device of FIG. 1, in accordance with an aspect of the present invention;

FIG. 4 is another side view of the intramedullary device of FIG. 1, in accordance with an aspect of the present invention;

FIG. 5 is a cross section of the intramedullary device of FIG. 1 taken along line 5-5 of FIG. 1, in accordance with an aspect of the present invention;

FIG. 6 is a bottom view of the intramedullary device of FIG. 1, in accordance with an aspect of the present invention;

FIG. 7 is a partially exploded view of an intramedullary device, in accordance with an aspect of the present invention;

FIG. 8 is a side view of a dispersion device, in accordance with an aspect of the present invention;

FIG. 9 is a side view of the intramedullary device of FIG. 7 with the dispersion device partially inserted into the intramedullary nail, in accordance with an aspect of the present invention;

FIG. 10 is a cross section of the intramedullary device of FIG. 7 with the dispersion device partially inserted into the intramedullary nail taken along line 10-10 of FIG. 9, in accordance with an aspect of the present invention;

FIG. 11 is a side view of the intramedullary device of FIG. 7 with the dispersion device fully inserted into the intramedullary nail, in accordance with an aspect of the present invention;

FIG. 12 is a cross section of the intramedullary device of FIG. 7 with the dispersion device fully inserted into the intramedullary nail taken along line 12-12 of FIG. 11, in accordance with an aspect of the present invention;

FIG. 13 is an exploded view of another embodiment intramedullary device, in accordance with an aspect of the present invention;

FIG. 14 is an isometric view of a first segment of the intramedullary device of FIG. 1, in accordance with an aspect of the present invention;

FIG. 15 is an isometric view from the top of the first segment of the intramedullary device of FIG. 1, in accordance with an aspect of the present invention;

FIG. 16 is an isometric view of a delivery segment of the intramedullary device of FIG. 1 from the bottom, in accordance with an aspect of the present invention;

FIG. 17 is a side view of the delivery segment of the intramedullary device of FIG. 1, in accordance with an aspect of the present invention;

FIG. 18 is an isometric view of the delivery segment of the intramedullary device of FIG. 1 from the top, in accordance with an aspect of the present invention;

FIG. 19 is a side view of a second segment of the intramedullary device of FIG. 1, in accordance with an aspect of the present invention;

FIG. 20 is an isometric view of the second segment of the intramedullary device of FIG. 1 taken from the top, in accordance with an aspect of the present invention;

FIG. 21 is an isometric view of an extension segment of the intramedullary device of FIG. 13 from a proximal end, in accordance with an aspect of the present invention;

FIG. 22 is an isometric view of the extension segment of the intramedullary device of FIG. 13 from a distal end, in accordance with an aspect of the present invention;

FIG. 23 is a side view of a shuttle of the dispersion device of FIG. 8, in accordance with an aspect of the present invention;

FIG. 24 is a side isometric view of the shuttle of the intramedullary device of FIG. 8, in accordance with an aspect of the present invention;

FIG. 25 is an isometric view of the shuttle of the intramedullary device of FIG. 8 taken from the front, in accordance with an aspect of the present invention;

FIG. 26 is a side view of the shuttle of the intramedullary device of FIG. 8 including two o-rings, in accordance with an aspect of the present invention;

FIG. 27 is an isometric view of a delivery tube of the dispersion device of FIG. 8, in accordance with an aspect of the present invention; and

FIG. 28 is a cross section of an intramedullary device inserted into a patient's bone and fixed with a fixation system, in accordance with an aspect of the present invention.

### DETAILED DESCRIPTION

In this application, the words proximal, distal, anterior, posterior, medial and lateral are defined by their standard usage for indicating a particular part or portion of a bone or prosthesis coupled thereto, or directional terms of reference, according to the relative disposition of the natural bone. For example, “proximal” means the portion of a bone or prosthesis nearest the torso, while “distal” indicates the portion of the bone or prosthesis farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body.

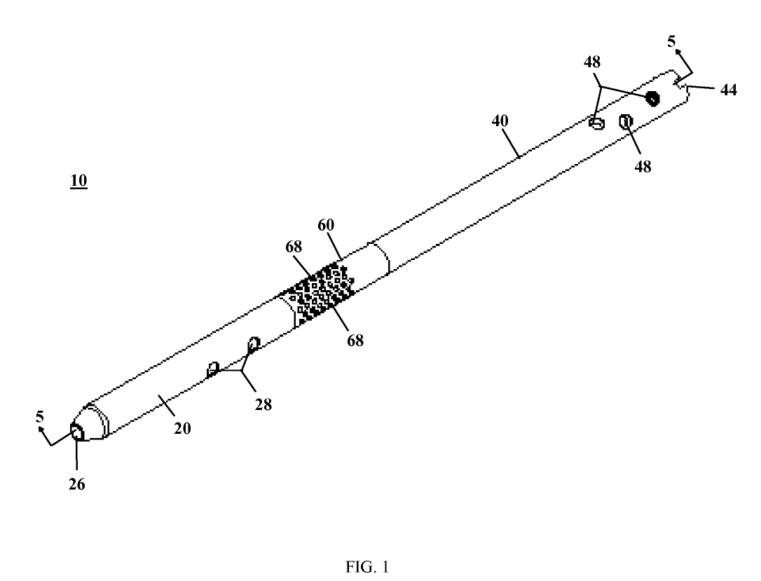

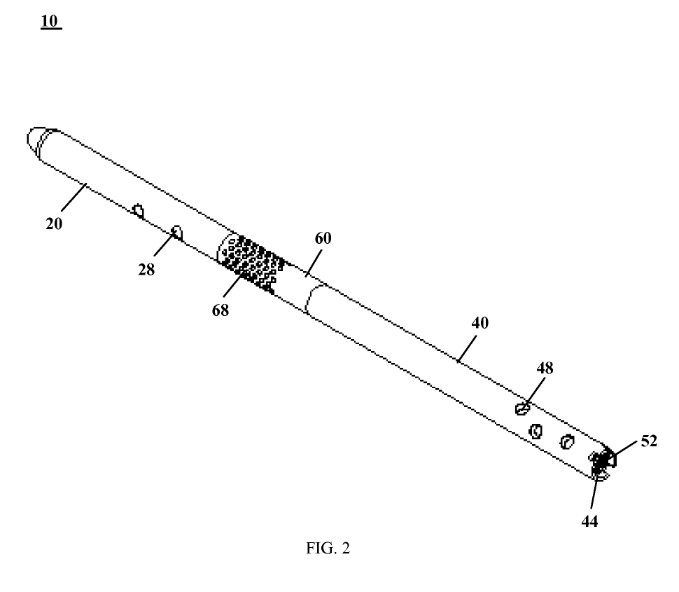

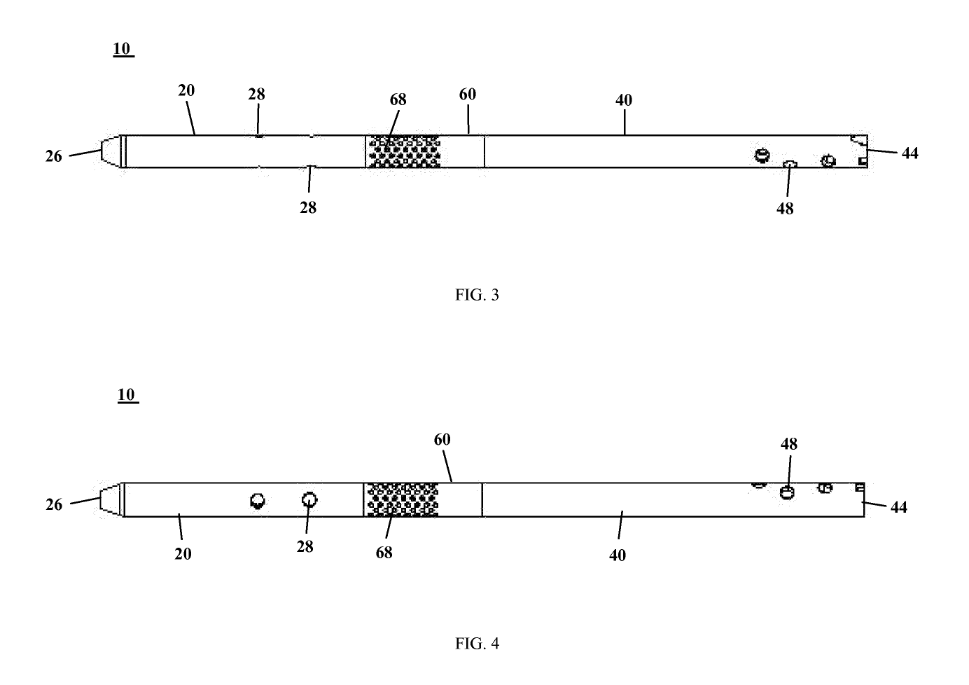

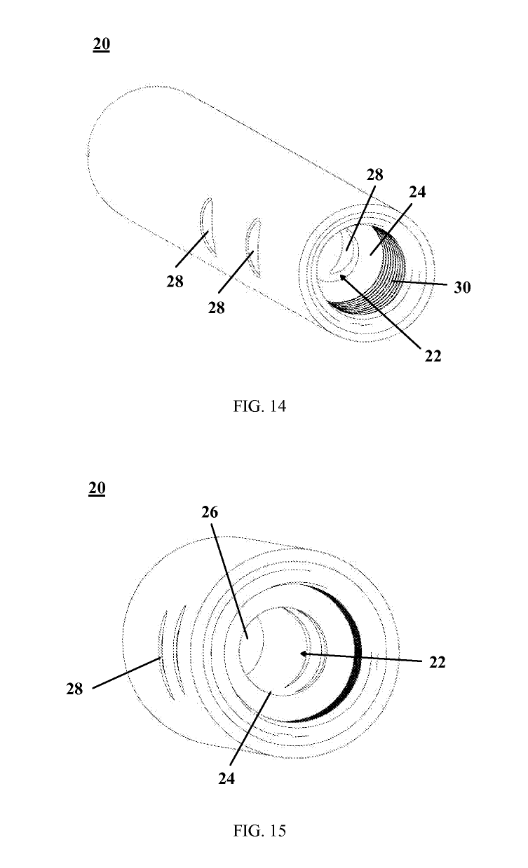

Referring to the drawings, wherein like reference numerals are used to indicate like or analogous components throughout the several views, and with particular reference to FIGS. 1-6, there is illustrated an exemplary embodiment intramedullary device or nail 10. The intramedullary nail 10 may include a first non-delivery segment 20, a second non-delivery segment 40, and a delivery segment or dispersion segment 60 connecting the first segment 20 and second segment 40. The first non-delivery segment 20, second non-delivery segment 40, and delivery segment 60 are made of a biomedical material, for example, a metal, such as, titanium, a composite, or bioabsorbable materials. The biomedical material may be impregnated with antimicrobial agents, for example, silver coatings, to help prevent infection. As best seen in FIGS. 14 and 15, the first segment 20 includes an interior channel 22 along the longitudinal axis of the first segment 20. The channel 22 travels from a first opening 24 to a second opening 26 (see FIG. 15). The first segment 20 also includes at least one through hole 28 perpendicular to the channel 22 for inserting at least one fastener to secure the intramedullary nail 10 to a bone. In the depicted embodiment there are two through holes 28, although it is also contemplated that the number of through holes 28 may range from, for example, two to four through holes. The proximal end of the first segment 20 may also include a fastening mechanism 30. The fastening mechanism 30 may be, for example, a female threaded section as depicted in the present invention, alternative fastening mechanisms 30, for example, a quick lock, snap fit, snap lock mechanisms, Morse tapers, and the like, are also contemplated. The fastening mechanism 30 may be reversible or non-reversible, in the present invention the fastening mechanism 30 is preferably non-reversible. The first segment 20 may have a length ranging from about, for example, 1 inch to about 6 inches with an inner diameter ranging from, for example, about 5 mm to about 10 mm, and an outer diameter ranging from, for example, about 9 mm to about 15 mm.

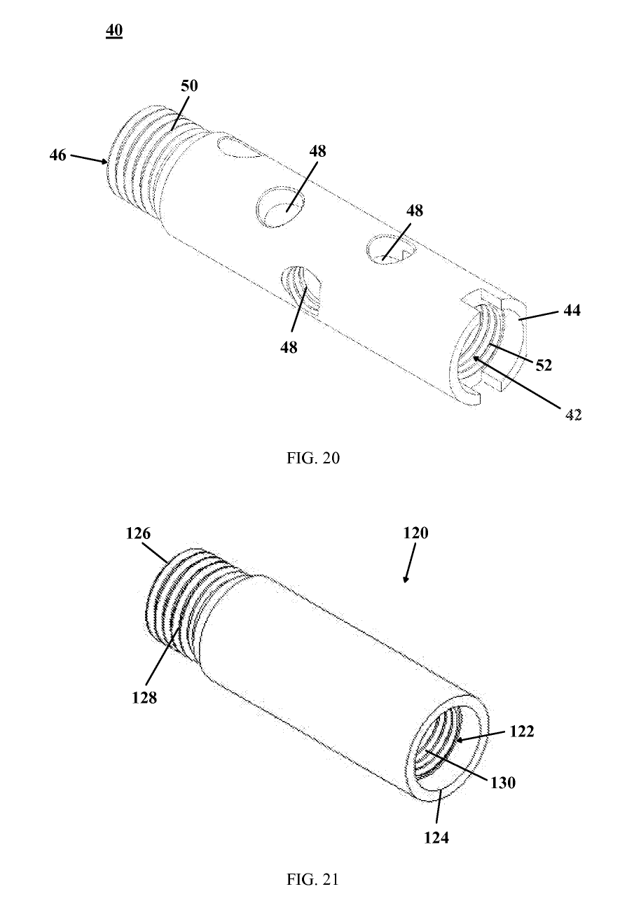

Referring now to FIGS. 19 and 20, the second segment 40 is depicted and includes an interior channel 42 (see FIG. 20) along the longitudinal axis of the second segment 40. The channel 42 travels from a first opening 44 to a second opening 46. The second segment 40 also includes at least one through hole 48 perpendicular to the channel 42 for inserting at least one fastener to secure the intramedullary nail 10 into the bone. In the illustrated embodiment there are three through holes 48, although it is also contemplated that the number of through holes 48 may range from, for example, two to four through holes. The distal end of the second segment 40 may also include a first fastening mechanism 50. The first fastening mechanism 50 may be, for example, a male threaded section as depicted in the present invention, although alternative fastening mechanisms 50, for example, a quick lock, snap fit, snap lock mechanisms, Morse tapers, and the like, are also contemplated. The fastening mechanism 50 may be reversible or non-reversible, in the present embodiment, the fastening mechanism 50 is preferably non-reversible. The proximal end of the second segment 40 may include a second fastening mechanism 52. The second fastening mechanism 52 may be, for example, a female threaded section as depicted in the present invention. Alternative second fastening mechanisms 52 are also contemplated, such as quick lock, snap fit, snap lock mechanisms, Morse tapers, and the like. The second fastening mechanism 52 may be reversible or non-reversible and second the fastening mechanism 52 is preferably non-reversible in the illustrated embodiment. The second segment 40 may have a length ranging from, for example, about 1 inch to about 6 inches with an inner diameter ranging from, for example, about 5 mm to about 10 mm and an outer diameter ranging from, for example, about 9 mm to about 15 mm.

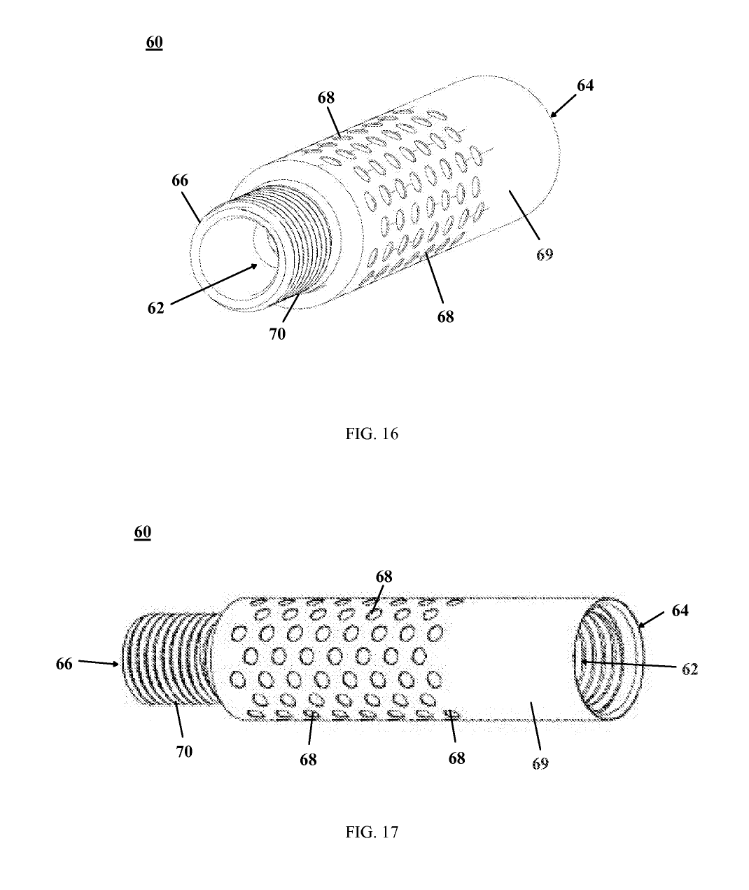

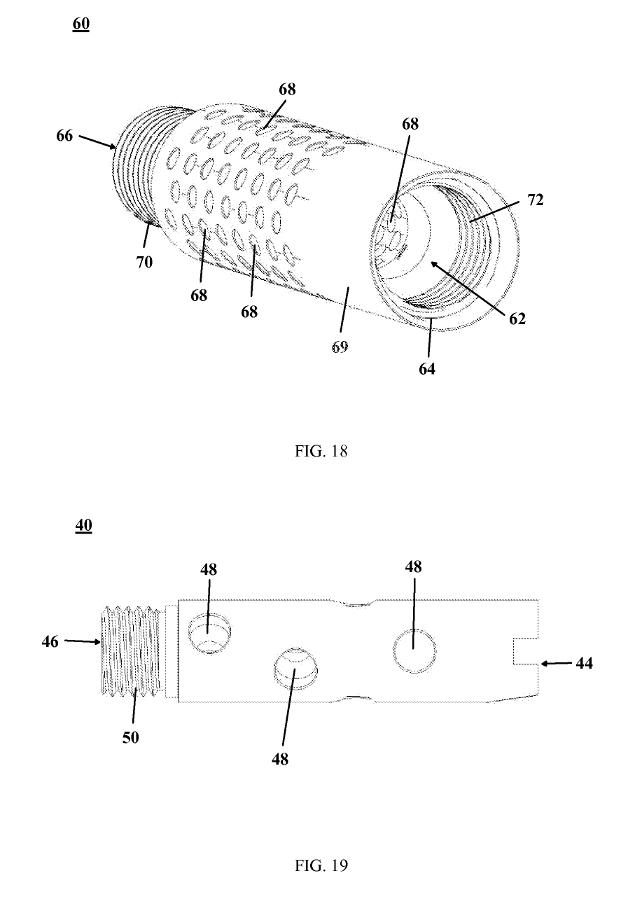

The delivery segment 60 is best seen in FIGS. 16-18 and includes an interior channel 62 along the longitudinal axis of the delivery segment 60. The channel 62 travels from a first opening 64 to a second opening 66. The delivery segment 60 also includes a plurality of through holes 68 passing from the channel 62 to an outer surface 69 of the delivery segment 60. The plurality of through holes 68 allow materials, for example, biomedical materials, to exit the intramedullary nail 10 into the location of a bone deficiency into the surrounding tissues or remaining bone. The distal end of the delivery segment 60 may also include a first fastening mechanism 70. The first fastening mechanism 70 may be, for example, a male threaded section as shown in the depicted embodiments. Alternative fastening mechanisms 70 are also contemplated, for example, quick lock, snap fit, snap lock mechanisms, Morse tapers, and the like. The fastening mechanism 70 is preferably non-reversible, although reversible fastening mechanisms 70 are also contemplated. The proximal end of the delivery segment 60 may include a second fastening mechanism 72. In the illustrated embodiment the second fastening mechanism 72 may be, for example, a female threaded section, although alternative embodiments are contemplated including, for example, quick lock, snap fit, snap lock mechanisms, Morse tapers, and the like. The fastening mechanism 72 is preferably non-reversible, although reversible fastening mechanisms 72 are also contemplated. The delivery segment 60 of the intramedullary nail 10 is modular and may be customized to allow for delivery of biomedical material to a deficiency in the bone at any location. The delivery segment 60 may have a length ranging from, for example, about 1 inch to about 8 inches with an inner diameter ranging from, for example, about 3 mm to about 10 mm and more preferably from about 3 mm to about 7 mm, and an outer diameter ranging from, for example, about 9 mm to about 15 mm.

The intramedullary nail 10 may be assembled by the surgeon just prior to implantation and customized for the exact location of the site of a bone deficiency specifically for each patient, such as a deficiency due to cancer, significant trauma, bone loss or weakness. The surgeon may select a delivery segment 60 including the desired number and desired size of through holes 68 based on the material(s) to be injected into the bone deficiency and the desired rate of injection. Once the delivery segment 60 is selected the first non-delivery segment 20 and second non-delivery segment 40 may be selected to position the delivery segment 60 at the location of the bone deficiency or fracture. The first segment 20, second segment 40, and delivery segment 60 may be selected and secured together in any order. The first non-delivery segment 20 may be smaller than, larger than, or the same size as the second non-delivery segment 40 to allow for placement of the delivery segment 60 anywhere along the intramedullary nail 10. Further, additional delivery segments 60 may be placed along the intramedullary nail 10 if necessary to disperse biomedical materials to multiple locations within the bone.

After the segments 20, 40, and 60 are selected the first segment 20 may be secured to the delivery segment 60 at a distal end and the second segment 40 may be secured to the delivery segment 60 at the proximal end. By way of specific example, the male threaded section of the delivery segment's first fastening mechanism 70 will be inserted into the female threaded section of the first segment's fastening mechanism 30. Then the male threaded section of the second segment's fastening mechanism 50 will be inserted into the female threaded section of the delivery segment's second fastening mechanism 72.

In another embodiment, where the first segment 20, second segment 40, and delivery segment 60 are Morse tapers, the fastening mechanism 70 of the delivery segment 60 will include a tapered distal end (not shown). The tapered distal end (not shown) of the delivery segment 60 may be placed in the first opening 24 of the first segment 20 which may also be tapered from the first opening 24 to the second opening 26. In addition, the second segment 40 may be tapered from the first opening 44 to the second opening 46. The tapered distal end (not shown) of the fastening mechanism 50 of the second segment 40 may be inserted into the first opening 64 of the delivery segment 60. Once the delivery segment 60 is inserted into the first segment 20 and the second segment 40 is inserted into the delivery segment 60 to form an intramedullary device 10 a force may be applied to the proximal and distal ends of the intramedullary device 10 to secure the first segment 20, second segment 40, and delivery segment 60 together. The force may be applied, for example, by a mechanical press, a hammer, or other known methods of securing Morse taper components together.

When multiple delivery segments 60 are placed along the intramedullary nail 10 a center non-delivery segment, not shown, may be inserted between the multiple delivery segments 60 and the first segment 20 will be attached to the delivery segment 60 located at the distal end and the second segment 40 will be attached to the delivery segment 60 located at the proximal end of the intramedullary nail 10. The resulting intramedullary nail 10 places the delivery segments 60 precisely where the surgeon wants them for delivery of biomedical materials to the site of the bone deficiency.

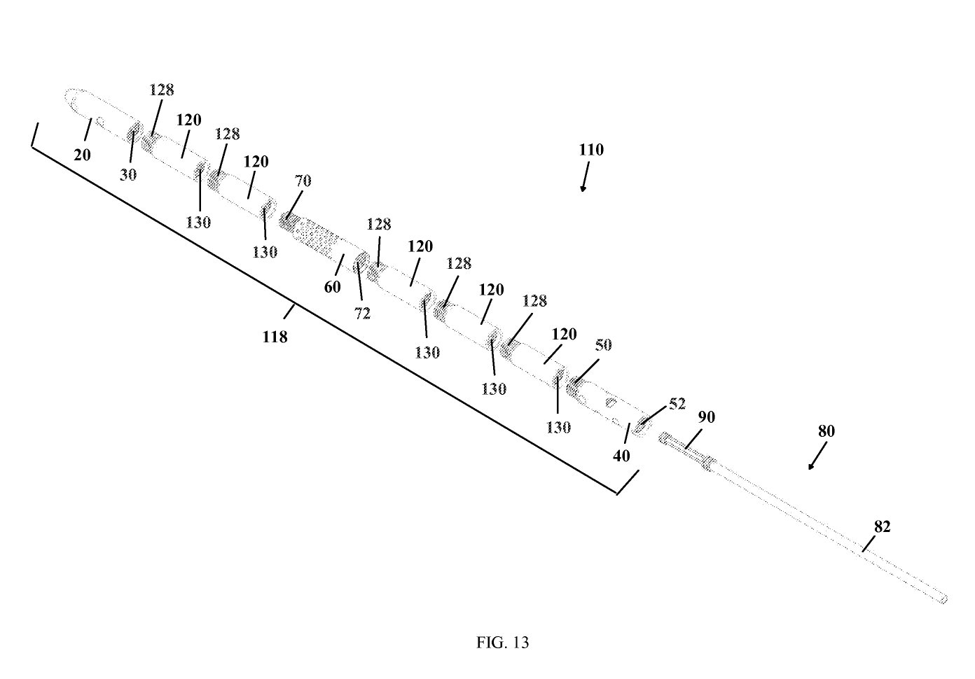

If additional length is needed for the intramedullary nail 10 for the embodiment depicted in FIG. 1 or the nail is comprised of extension segments as illustrated in FIG. 13, extension segments 120, may be used. The extension segments 120, as shown in FIGS. 13, 21, and 22, may be inserted between the first segment 20 and the delivery segment 60, between the delivery segment 60 and the second segment 40. These extension segments may range from, for example, approximately 1 inch to 6 inches and are more preferably about one and a half inch segments. The resulting intramedullary nail 10 will range from, for example, approximately 10 inches to 40 inches. In alternative embodiments, extension segments 120 may also be attached at the proximal end of the second segment 40.

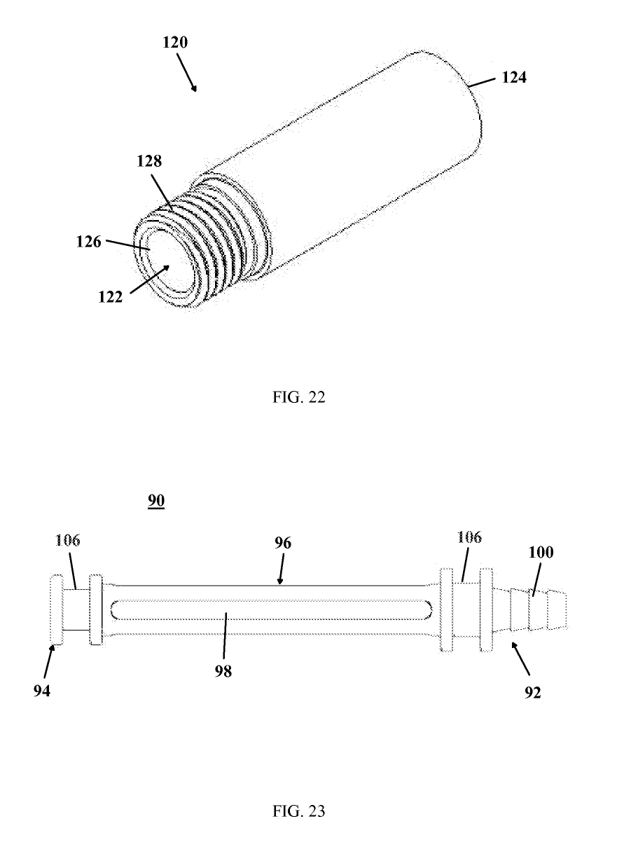

Referring now to FIGS. 21-22, the extension segments 120 are illustrated. The extension segments 120 include an interior channel 122 along the longitudinal axis of the extension segments 120. The channel 122 travels from a first opening 124 to a second opening 126. The distal end of the extension segments 120 may also include a first fastening mechanism 128. The first fastening mechanism 128 may be, for example, a male threaded section as depicted in the present invention, alternative fastening mechanisms 128, for example, a quick lock, snap fit, snap lock mechanisms, Morse tapers, and the like, are also contemplated. The first fastening mechanism 128 may be reversible or non-reversible, in the present invention the fastening mechanism 128 is preferably non-reversible. The proximal end of the extension segments 120 may include a second fastening mechanism 130, as shown in FIG. 21. In the illustrated embodiment the second fastening mechanism 130 may be, for example, a female threaded section, although alternative embodiments are contemplated including, for example, quick lock, snap fit, snap lock mechanisms, Morse tapers, and the like. The second fastening mechanism 130 is preferably non-reversible, although reversible fastening mechanisms 130 are also contemplated. The extension segments 120 of the intramedullary nail 10 are modular and may be inserted anywhere along the nail where additional length is needed. The extension segments 120 are generally inserted between the first non-delivery segment 20 and the delivery segment 60 and between the delivery segment 60 and the second non-delivery segment 40.

Any additional delivery segments 60 or extension segments of the intramedullary nail 10 may also include a fastening or locking mechanism, not shown, that allows for the locking and unlocking of the segments 20, 40, and 60 of the intramedullary nail 10 relative to each other. The locking mechanism may be, for example, a quick lock, snap fit, snap lock mechanism, Morse taper, and the like, which allows for the segments 20, 40, and 60 and extension segments 120, if used, to be secured together to prevent the segments 20, 40, 60, and 120 from disconnecting while implanted in a patient.

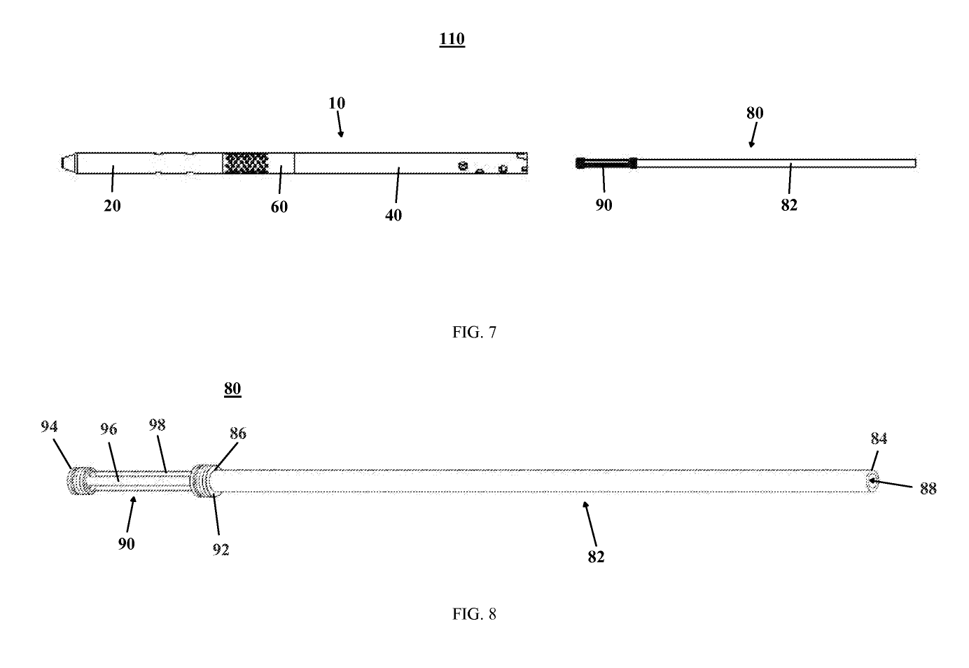

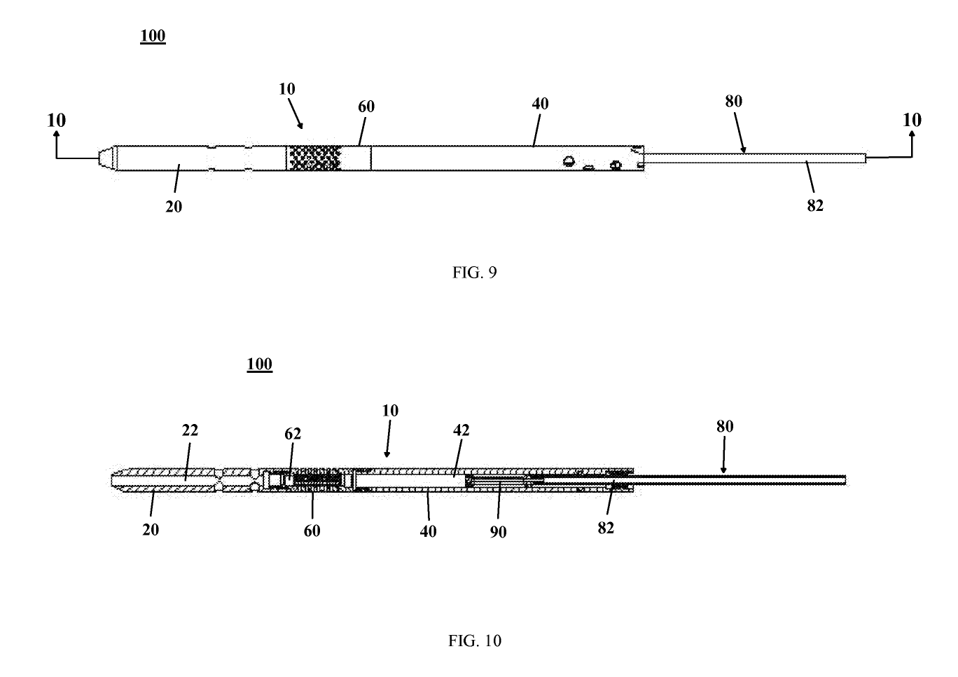

Referring now to FIGS. 7 and 9-12, a modular intramedullary device or nail system 110 is shown. The modular intramedullary device system 110 includes a dispersion device 80 and the intramedullary nail 10. The intramedullary nail 10, described in greater detail above, provides for stabilization of the bone, while the dispersion device 80, described in greater detail below, allows for the precise placement of biomedical materials within the bone to augment the stabilization. The dispersion device 80 may also accept instrumentation to assist a surgeon determine movement and healing of the bone at the site of the bone deficiency.

An alternative modular intramedullary device system 110 is illustrated in FIG. 13. The alternative modular intramedullary device system 110 includes a dispersion device 80 and the intramedullary nail 118. The intramedullary nail 118 may be assembled by the surgeon just prior to implantation and customized for the exact location of the site of a bone deficiency specifically for each patient, such as a deficiency due to cancer, significant trauma, bone loss or weakness. The surgeon may select a delivery segment 60 including the desired number and desired size of through holes 68 based on the biomedical material(s) to be injected into the bone deficiency and the desired rate of injection. Once the delivery segment 60 is selected a first non-delivery segment 20 and a second non-delivery segment 40, as well as the desired number of extension segments 120 may be selected to position the delivery segment 60 at the location of the bone deficiency or fracture. As illustrated in the depicted embodiment of FIG. 13, two extension segments 120 are connected to the distal end of the delivery segment 60 before the first non-delivery segment 20 is attached. On the proximal end of the delivery segment 60 three extension segments 120 are connected prior to securing the second non-delivery segment 40. Any number of extension segments 120 may be selected for insertion between the first segment 20 and the delivery segment 60 and between the delivery segment 60 and the second segment 40 to allow for placement of the delivery segment 60 anywhere along the intramedullary nail 118. Further, additional delivery segments 60 may be placed along the intramedullary nail 118 if necessary to disperse biomedical materials to multiple locations within the bone.

With continued reference to FIGS. 7 and 9-12, after the segments 20, 40, and 60 are selected the first segment 20 may be secured to the delivery segment 60 at a distal end and the second segment 40 may be secured to the delivery segment 60 at the proximal end. Specifically, the male threaded section of the delivery segment's first fastening mechanism 70 will be inserted into the female threaded section of the first segment's fastening mechanism 30. Then the male threaded section of the second segment's fastening mechanism 50 will be inserted into the female threaded section of the delivery segment's second fastening mechanism 72. When multiple delivery segments 60 are placed along the intramedullary nail 10 a center non-delivery segment, not shown, may be inserted between the multiple delivery segments 60 and the first segment 20 will be attached to the delivery segment 60 located at the distal end and the second segment 40 will be attached to the delivery segment 60 located at the proximal end of the intramedullary nail 10. The resulting intramedullary nail 10 places the delivery segments 60 precisely where the surgeon wants them for delivering biomedical materials to the site of the bone deficiency.

As seen in FIG. 8, the dispersion device 80 includes a tube 82 and a shuttle or dispensing member 90. In order to deliver materials, for example, irrigation or cleaning fluids, bone regenerative materials, or bone cement, the dispersion device 80 is inserted into the intramedullary nail 10. The dispensing member 90 is inserted into the first opening 44 and slid into alignment with the delivery segment 60. The interior channel 62 may include a stop member (not shown) at its distal end to stop the dispensing member 90 in the desired location for release of the delivery materials into the bone fracture. Alternatively, the channel 22 may include a stop member (not shown) at its proximal end to stop the dispensing member 90 in the desired location for release of the delivery materials into the bone fracture. The dispersion device 80 may be made of, for example, a polymer or composite material. The polymer material used for the dispersion device 80 may be a long term polymer material or a resorbable material.

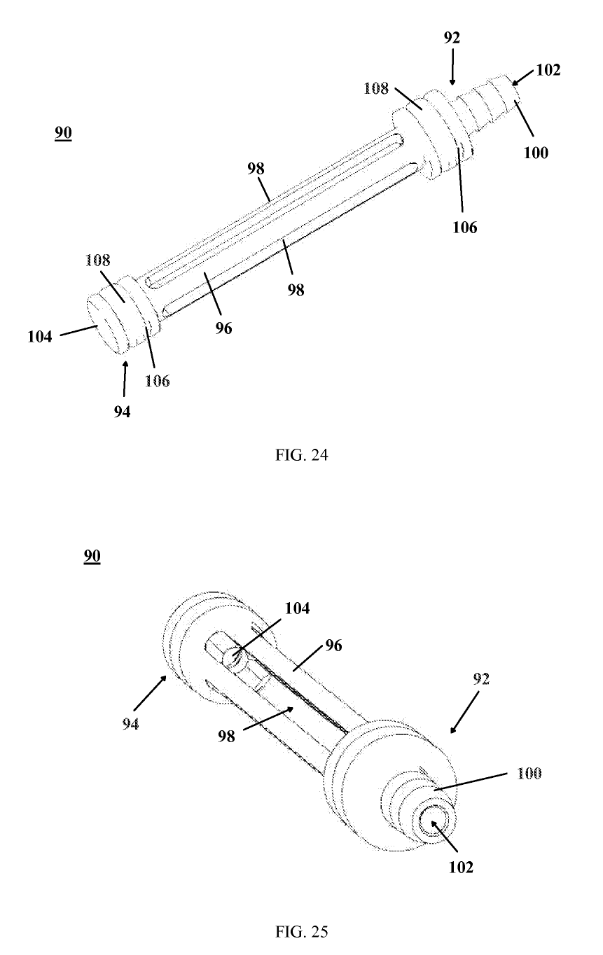

As illustrated in FIGS. 23-26, the dispensing member 90 includes a proximal end 92 and a distal end 94 connected by at least one center member 96 with at least one dispersion opening 98. In the depicted embodiment, there are four center members 96 connecting the proximal end 92 and the distal end 94. The four center members 96 create four dispersion openings 98 for releasing the delivery materials into the bone fracture site. In the illustrated embodiment, the center members 96 provide large dispersion openings 98 to enable a full 360 degree dispersion of materials through the delivery segment 60. The dispensing member 90 also includes an attachment portion 100 at the proximal end 92 to connect the dispensing member 90 and the tube 82. In the depicted embodiment, the attachment portion 100 is a stepped up connector section creating a tight fit when the tube 82 is inserted over the attachment portion 100. The attachment portion 100 includes an opening 102 (see FIG. 25) allowing for delivery materials to pass from the tube 82 into the dispensing member 90 for delivery out of the dispersion openings 98. The distal end 94 may include an optional opening 104, as depicted in FIGS. 24-25. Further, the dispensing member 90 may also include at least one groove 106 (see FIG. 23) for mating with at least one o-ring 108 (see FIG. 24). In the depicted embodiment, there are two grooves 106 and two o-rings 108 with a first groove 106 and o-ring 108 at the proximal end 92 of the dispensing member 90 and a second groove 106 and o-ring 108 at the distal end 94 of the dispensing member 90. Further, a sealing mechanism (not shown) may be provided to seal the ends of the dispensing member 90. The sealing mechanism may be a ring locking mechanism, wherein the proximal and distal ends each include a ring of material which has a slightly larger diameter than the inner diameter of the nail 10. The dispensing member 90 with the sealing mechanism may be snapped into the nail 10 to prevent fluids and in some case materials from flowing past the proximal and distal ends including the ring locking mechanisms.

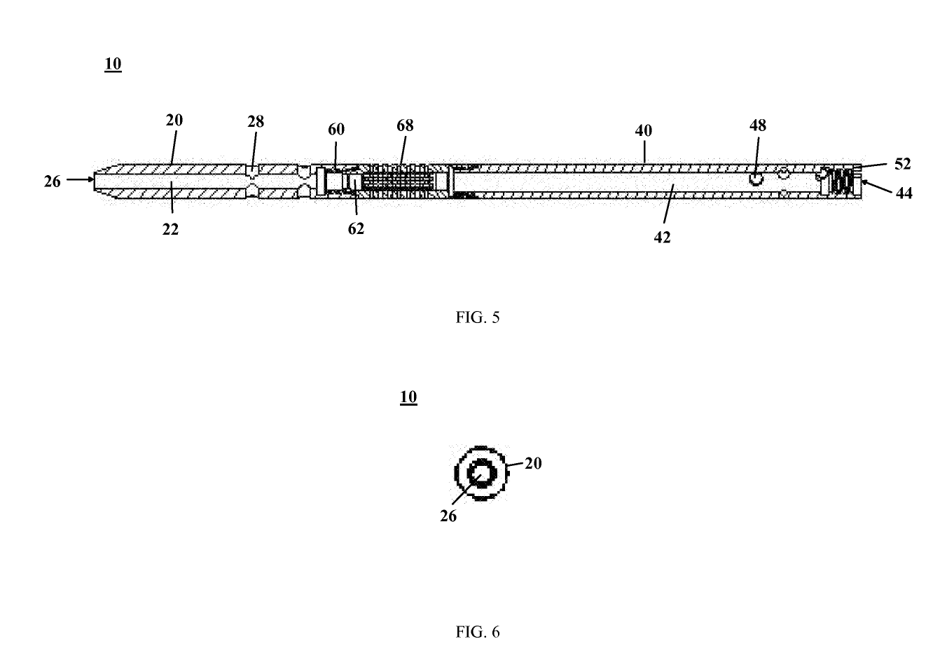

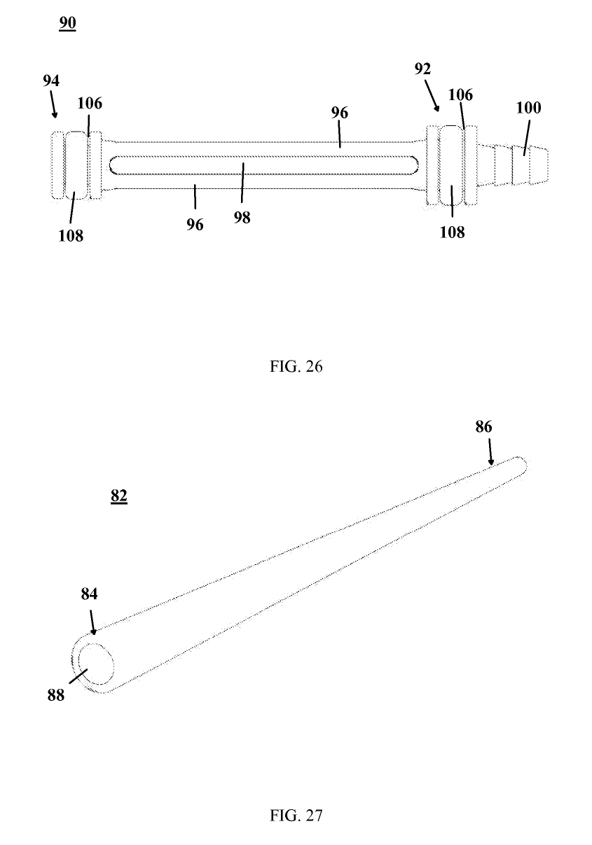

Referring now to FIG. 27, the tube 82 is illustrated and includes a proximal end 84 and a distal end 86 with an interior channel 88. The channel 88 runs along the longitudinal axis of the tube 82 from the proximal end 84 to the distal end 86. The distal end 86 of the tube 82 mates with the attachment portion 100 (see FIG. 26) of the dispensing member 90 to create the dispersion device 80, as shown in FIG. 8. Once the tube 82 and dispensing member 90 are secured together, a material injection system, not shown, may be secured to the proximal end 84 of the tube 82 for dispensing delivery materials into the dispersion device 80. The material injection system may include, for example, a syringe system, a one-way pump, a cement gun, an external pump and suction or pump and valve system, or the like, for dispensing delivery materials into the tube 82. The syringe system, one-way pump system, and cement gun are preferably used for dispersion of bone regenerative materials and bone cement through the delivery segment 60. The external pump and suction or pump and valve system is preferably used for irrigating or cleaning the area of bone deficiency by pumping a fluid to clean the wound into the dispersion device 80 and the extracting the fluid from the wound using suction to pull the fluid out of the nail 10 between the channels 62 and 42 (see FIG. 5) and the exterior of tube 82. The material injection system may be secured to the proximal end 84 of the tube 82 by a locking mechanism (not shown). The locking mechanism may be a threaded system or a quick connect-disconnect system.

Alternative dispersion devices 80 are also contemplated such as using a capsule system (not shown) that would be inserted into the first opening 44 of the second segment 40 (see FIG. 5). The capsule system would travel through the channel 42 and into the channel 62 in the delivery segment 60. Once in the desired position in the delivery segment 60 the capsule could be pierced to release the delivery material. The capsule could be pierced by a puncture tool (not shown), such as a sharp protrusion, within the channel 62 or alternatively the capsule could be pierced manually by inserting a puncture tool or a sharp instrument down the channel 42 of the second segment 40 until the instrument contacted the capsule and pierced it to release the dispersion materials. Another alternative dispersion devise 80 may include a tube 82 and an instrument (not shown) for deploying the biomedical material in the tube 82. The tube 82 may be, for example, flexible or rigid. The tube 82 may be inserted into the first opening 44 of the second segment 40 (see FIG. 5) and pushed into the nail 10 along the interior channel 42, 62 until the distal end 86 of the tube 82 engages at least a portion of the delivery segment 60. Then a delivery tool (not shown) may be inserted into the channel 88 at a proximal end 84 of the tube 82 to dispense the delivery material through the through holes 68 to the bone. Alternatively, the delivery tool (not shown) may be connected to the tube 82 at a proximal end to apply pressure through the tube causing the delivery material to dispense from the tube 82 into the delivery segment 60 and out the through holes 68 to the bone. The delivery tool may be, for example, a plunger, bougie, or the like. Each dispersion device 80 can prevent the delivery materials that are injected into the intramedullary device system 110 from flowing down the nail where the delivery materials are not needed. One method to prevent the delivery materials from flowing past the delivery segment 60 is by pressurizing the material injection system thereby forcing the delivery materials out of the plurality of holes 68. Another method to prevent the materials from flowing into channels 22 or 42 is to include a sealing mechanism (not shown) which seals the ends of the dispensing member 90.

The intramedullary device system 110 may be used to providing stabilization of a bone and limb, reconstruction of soft tissue defects, and the precise placement or delivery of materials within the bone to augment the stabilization by stimulating bone formation and providing a scaffold for bone formation. For example, bone cement may be delivered to bones that have been weakened or removed by cancer to fill the deficiencies in the bone. Alternatively materials to promote bone formation and healing may be delivered where bone is missing. Additional uses of the intramedullary device system 110 include but are not limited to irrigating bones and surrounding soft tissues when there are open and contaminated wounds, for example, in high energy injuries such as blast injuries or due to other trauma. Further, the intramedullary device system 110 also allows for bone regeneration materials, such as growth stimulators, to be placed at a site of fracture or bone loss to stimulate bone formation around the nail at that site. Growth stimulators may include, for example, platelet derived growth factor (“PDGF”), vascular endothelial growth factor (“VEGF”) and epidermal growth factor, which may be used to initiate healing by promoting cell replication and repair. The intramedullary device system 110 may also be used to deliver bone material, for example from the reamings, or from allograft preparations that stimulate bone formation from the surrounding tissue. Yet further, the intramedullary device system 110 may also be used to provide drugs or chemicals to the bone or tissues within the bone. The drugs or chemicals could be used to prevent or treat infection or to provide drugs or medically active chemicals to the entire body from a reservoir within the intramedullary device system 110. The intramedullary device system 110 may also be used to treat bones that have a regular bone fracture, as well as bones that are at risk from fracturing by allowing the placement of materials or substances that will strengthen or improve the bone's response to physiologic activities.

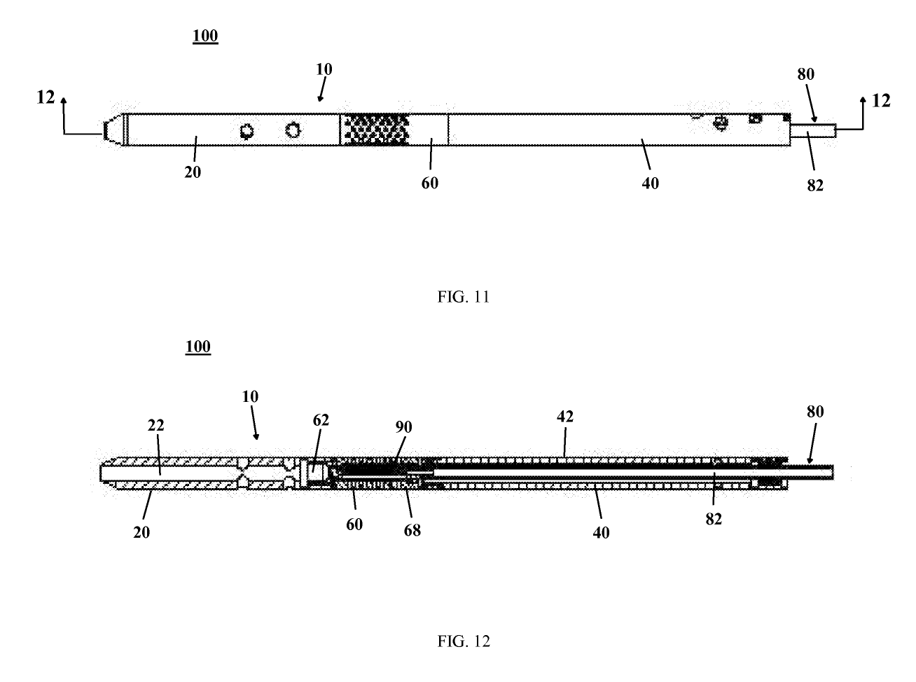

A surgical method for implanting an intramedullary device includes obtaining an intramedullary device system 110 for insertion into a patient's bone. The bone is then prepared for insertion of the intramedullary device system 110 by inserting a guidewire into the bone then drilling over the guidewire to create a canal for the nail 10. The nail 10 of the intramedullary device system 110 is then inserted into the canal created in the bone. The nail 10 is positioned so the delivery segment 60 is located at the bone deficiency. The dispersion device 80 is then inserted into an interior longitudinal channel created by channels 22, 42 and 62 (see FIG. 5) in the nail 10 until the distal end of the dispensing member 90 is aligned with the distal end of the delivery segment 60 (see FIGS. 11-12). Then material is dispersed from the dispersion device 80 at the dispensing member 90 through the delivery segment 60 to the damaged portion of the bone.

For example, once the intramedullary device system 110 is assembled by the surgeon and inserted into the bone of the patient, the device system 110 can be used as an irrigation device to the wound where both the bone and the surrounding soft tissue envelope has been injured.

It is accepted medical practice that the treatment of open wounds involving bone fractures requires the patient to be taken to an operating facility where the wound can be surgically treated to remove all visible foreign material, all dead tissues and dead bone. The wound may be washed with fluids during or after this debridement. The usual practice is then to either close the wound or apply a sponge and covering to the wound and apply suction to remove fluids from the injured area.

The intramedullary device system 110 may provide, for example, ongoing fluid lavage to both washout the wound and remove microscopic foreign material, bacteria and other noxious organisms and blood clots that may harbor and encourage growth of bacteria. The fluids that are delivered to the injured area may also contain antibiotics and antiseptics that will further inhibit growth of bacteria. The use of a low pressure pulsatile system for fluid delivery is unique in the application of pressure allows for the soft tissues to be lightly distended so that fluid flows to all parts of the wound, and then allows for the fluid to be removed, so improving the washout ability for all materials. The pulsatile pressure is also beneficial to the soft tissues as it may prevent contractures of the soft tissues, keeping them pliable and elastic while healing occurs. The fluid management system here described may also speed the resolution and prevention of infection, which is the main early complication of traumatic open wounds to long bone fractures.

An alternative embodiment of the dispersion device 80 allows for an early irrigation system such as a tubing apparatus (not shown) to be inserted through the incision used to insert the nail and then passed into the end of the nail closest to the skin wound. The tubing apparatus is inserted into the nail 10, in such a fashion that there is a water tight seal at the distal or far end of the delivery segment 60, and a watertight seal at the proximal or near end of the delivery segment 60. This allows for irrigation of only the injured part of the bone and soft tissues. The tubing apparatus consists of two passageways within the tube, one having a large bore and the second having smaller bore. The fluid pressure of the inlet and exit fluid of the tube will be monitored externally near the proximal end of the tube (given the low flow rates, these pressure readings outside of the nail will be close approximations of internal pressures). The large bore passageway is the outlet for the fluids, and the fluids flow from the delivery segment 60 up the tubing to a connector out of the patient. There the fluids may flow over or through material which gathers bacterial and fungal DNA and RNA for analysis at a laboratory to determine the type of infection that might be present within the patient. The fluids then flow to a container for disposal. Alternatively the fluids may flow from the delivery segment 60 up the tubing to a connector out of the patient and to a container for disposal. The tubing apparatus of the dispersion device 80 is a closed system.

The smaller bore passageway is the inflow for fluids and is connected to a pump which applies a pulsating pressure, with that pressure being adjustable by attending health care personnel. The source of fluids for the pump consists of a regular IV bag in which different chemicals or antibiotics can be placed on the orders of a medical doctor. There is a closed loop system of controls from the pressure monitors within the delivery segment 60 of the irrigation system to the pump which controls the pressure for the inlet line. Pressures within the delivery segment 60 cannot exceed pressure limits set by the attending health care personnel. The health care personnel can control the pressure and the amount of fluids dispensed by the pump.

After irrigation of the wound or dispersion of desired fluids, the dispersion device 80 may then be removed and the nail 10 secured to the bone by inserting pins or other bone fastening mechanisms through the through holes 28 of the first segment 20 (see FIG. 1). Alternatively, the first segment 20 may have been secured to the bone prior to insertion of the delivery segment 60 into the nail 10. Once all material has been inserted into the bone for the present procedure the second segment 40 may optionally be secured to the bone by inserting pins or other bone fastening mechanisms into through holes 48 (see FIG. 1).

If the surgeon intends to deliver additional materials to the bone deficiency the surgeon may decide not to secure the second segment 40 to the bone to provide continued access to the nail 10 through the healing process. However, if the surgeon will be allowing the patient to perform weight bearing activities on the bone which received the nail 10, the second segment 40 should be secured to the bone. If the second segment 40 is secured to the bone using bone fastening mechanisms (not shown) which traverse the channel 42 and additional material is to be inserted into the nail 10 at a later date, the threaded pins or other bone fastening mechanisms would have to be removed prior to insertion of the dispersion device 80 into the nail 10. After the bone deficiencies have been completely stabilized or healed, the intramedullary nail 10 may be removed from the patient's bone.

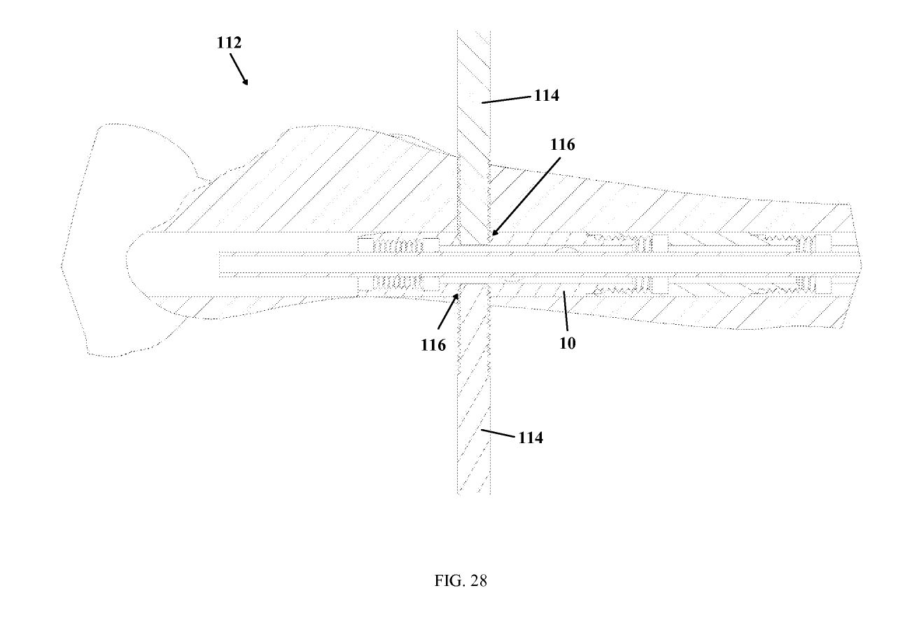

As the locking or transfixion screws used to stabilize the nail 10 to the bone described above would occupy the inside of the nail 10 and interfere with the passage of materials down the nail 10 through channel 42, an alternative fixation system 112, shown in FIG. 28, may be used to maintain access to the delivery segment 60 throughout the healing process without having to insert and remove locking screws. The fixation system 112 allows for temporary locking screws 114 to be placed using standard guides that fit the proximal end of the nail 10, whereby the holes may be drilled through the bone towards the nail 10. However, instead of the holes being present in the nail 10, captured gimbals are provided with holes in them or small tapered detents in the outer surface of the nail 10. The temporary locking screws 114 will engage the gimbals or detents but do not enter the channel 42 of the nail 10, thereby leaving the inside channel 42 of the nail 10 free for passage by a dispersion device 80. The temporary locking screws 114 may be longer than the bones and may extend into the soft tissue and muscle surrounding the patient's bone, but are contained within the patient's skin. After irrigation is complete and new bone begins forming in the region of the fracture, the temporary fixation screws 114 can be removed and replaced with standard fixation screws as described above which are inserted into the through holes 48 (see FIG. 1) in the proximal end of the nail. The standard fixation screws will traverse the through holes 48, which may be tapped through holes. After the bone deficiencies have been completely stabilized or healed, the intramedullary nail 10 may be removed from the patient's bone.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. An intramedullary device, comprising:
a nail with a proximal end and a distal end, the nail comprising:
a first segment proximate the distal end;
a second segment proximate the proximal end; and
a delivery segment connecting the first segment and the second segment.

2. The intramedullary device of claim 1, wherein the nail further comprises an interior channel extending from the proximal end to the distal end and disposed within the first segment, the delivery segment, and the second segment.

3. The intramedullary device of claim 2, wherein the first segment comprises:
a first opening;
a second opening opposite the first opening, wherein the first opening and second opening are connected by the interior channel; and
at least one hole perpendicular to the interior channel.

4. The intramedullary device of claim 3, wherein the first segment further comprises a fastening mechanism adjacent to the first opening.

5. The intramedullary device of claim 4, wherein the second segment comprises:
a first opening;
a second opening opposite the first opening, wherein the first opening and second opening are connected by the interior channel; and
at least one hole perpendicular to the interior channel.

6. The intramedullary device of claim 5, wherein the second segment further comprises a fastening mechanism adjacent to the second opening.

7. The intramedullary device of claim 6, wherein the fastening mechanism of the first segment comprises at least one of a female section, a male section, a quick lock, a snap fit, and a snap lock mechanism and the fastening mechanism of the second segment comprises at least one of a female section, a male section, a quick lock, a snap fit, and a snap lock mechanism.

8. The intramedullary device of claim 7, wherein the female section and the male section of the first segment are threaded and the female section and male section of the second segment are threaded.

9. The intramedullary device of claim 6, wherein the delivery segment comprises:
a first opening;
a second opening opposite the first opening, wherein the first opening and second opening are connected by the interior channel; and
a plurality of holes passing from the channel to an exterior surface of the delivery segment.

10. The intramedullary device of claim 9, wherein the delivery segment further comprises:
a first fastening mechanism positioned near the second opening; and
a second fastening mechanism positioned near the first opening.

11. The intramedullary device of claim 10, further comprising:
at least one first extension segment connecting the first segment and the delivery segment; and
at least one second extension segment connecting the delivery segment and the second segment.

12. A delivery system, comprising:
a nail with a proximal end and a distal end, the nail comprising:
at least one first segment proximate the distal end;
at least one second segment proximate the proximal end; and
a delivery segment connecting the at least one first segment and the at least one second segment; and
a dispersion device with a proximal end and a distal end and configured to slidingly engage the nail.

13. The delivery system of claim 12, wherein the dispersion device is configured to pass through an interior channel of the nail to engage the delivery segment.

14. The delivery system of claim 13, wherein the dispersion device comprises:
a tube at a proximal end of the dispersion device; and
a dispensing member at a distal end of the dispersion device, wherein the dispensing member is coupled to the tube.

15. The delivery system of claim 14, wherein the dispensing member comprises:
a proximal end;
a distal end opposite the proximal end;
at least one center member connecting the proximal end and the distal end;
at least one dispersion opening configured to be parallel to the at least one center member; and
an attachment portion at the proximal end configured to engage the distal end of the tube.

16. The delivery system of claim 15, wherein the dispensing member further comprises:
at least one groove extending around at least a portion of the circumference of an exterior surface of the dispensing member; and
at least one sealing mechanism for engaging the at least one groove.

17. The delivery system of claim 13, wherein the dispersion device comprises:
a capsule of biomedical material; and
a puncture tool configured to engage the capsule when the dispersion device is positioned within the delivery segment.

18. An intramedullary device system, comprising:
a nail with a proximal end and a distal end, the nail comprising:
a first segment at the distal end;
a second segment at the proximal end;
a delivery segment medial to the first segment and the second segment;
a first plurality of extension segments connecting the first segment and the delivery segment; and
a second plurality of extension segments connecting the delivery segment and the second segment; and
a dispersion device configured to transport a biomedical material to be dispersed into a bone through an interior channel in the nail.

19. A surgical method for implanting an intramedullary device, comprising:
obtaining the intramedullary device, comprising:
a nail with a proximal end and a distal end, the nail comprising:
a first segment at the distal end;
a second segment at the proximal end;
a delivery segment connecting the first segment and the second segment; and
an interior channel extending through the first segment, the delivery segment, and the second segment; and
a dispersion device having a proximal end and a distal end and is configured to engage the delivery segment;
inserting the nail into a canal within a bone;
aligning the delivery segment with a damaged portion of the bone;
inserting the dispersion device into the interior channel of the nail, wherein the distal end of the dispersion device is aligned with a distal end of the delivery segment; and
dispensing a biomedical material through the dispersion device and delivery segment to the damaged portion of the bone.

20. A method of assembling an intramedullary device, comprising:
selecting a first segment;
selecting a delivery segment;
securing the delivery segment to a proximal end of the first segment;
selecting a second segment; and
securing the second segment to a proximal end of the delivery segment opposite the first segment.

21. The method of claim 20, further comprising:
selecting at least one first extension segment;
coupling the at least one extension segment between the first segment and the delivery segment;
selecting at least one second extension segment; and
coupling the at least one extension segment between the delivery segment and the second segment.

22. The method of claim 21, further comprising:
selecting a dispersion device with a length to extend into the delivery segment when inserted into a proximal end of the second segment.
